(19) United States
(12) Patent Application Publication
(10) Pub. No.: US 2023/0407321 A1
(43) Pub. Date: Dec. 21, 2023

(54) METHOD TO DELIVER CHEMICALS AND NUCLEIC ACIDS TO PLANT CELLS USING CELLULOSE NANOCRYSTALS AS A CARRIER

(71) Applicants: BOARD OF TRUSTEES OF THE UNIVERSITY OF ARKANSAS, Little Rock, AR (US); AUBURN UNIVERSITY, Auburn, AL (US)

(72) Inventors: Mariya Khodakovskaya, Little Rock, AR (US); Virginia Davis, Auburn, AL (US); Delaney Clouse, Wetumpke, AL (US); Kari Vinzant, Benton, AR (US)

(21) Appl. No.: 18/207,797

(22) Filed: Jun. 9, 2023

Related U.S. Application Data
(60) Provisional application No. 63/350,470, filed on Jun. 9, 2022.

(51) Int. Cl. C12N 15/82; A01N 25/24; A01N 37/38

(52) U.S. Cl. CPC C12N 15/8206; A01N 37/38; A01N 25/24

(57) ABSTRACT

A nanomaterial conjugate for delivering an active agent to a plant comprising a nanocarrier and an active agent linked to the nanocarrier. The nanocarrier is cellulose nanocrystal (CNC), and the nanocarrier delivers the active agent across cell walls of cells of the plant.

### CROSS-REFERENCE TO RELATED PATENT APPLICATIONS

This application claims priority to and the benefit of U.S. provisional patent application Ser. No. 63/350,470, filed Jun. 9, 2022, entitled “Method To Deliver Chemicals And Nucleic Acids To Plant Cells Using Cellulose Nanocrystals As A Carrier”, which is incorporated herein in its entirety by reference.

### STATEMENT AS TO RIGHTS UNDER FEDERALLY-SPONSORED RESEARCH

This invention was made with government support under Grant No. 2021-67022-33995 awarded by the National Institute of Food and Agriculture. The government has certain rights in the invention.

### FIELD OF THE DISCLOSURE

The disclosure relates generally to applications of cellulose nanocrystals (CNC) as delivery carriers for the transport of chemicals, e.g. growth regulators, pesticides, and large molecules including polynucleotides and polypeptides across plant cell walls.

### BACKGROUND OF THE DISCLOSURE

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the disclosure.

The need for more refined, efficient, and sustainable methods in food production is driven by several factors including a changing global climate, water shortages, and a rapidly growing global population. Nanotechnology has the potential to improve agricultural practices by enhancing crop growth, detecting pathogens, and hardening crops against abiotic stress.

Engineered nanomaterials such as carbon nanotubes (CNT) have shown promise as plant growth regulators, and nanocarriers for the delivery of biomolecules. However, there is a legitimate concern that the wide use of engineered nanomaterials in plant agriculture may result in nano-contaminated food products. When plants are exposed to nanomaterials through their roots or leaves, the nanoparticles can spread throughout the plant and reach reproductive tissues such as seeds and fruits, contaminating edible parts. While the concept of nanotechnology-enabled agriculture is exciting for researchers, it is important to consider and adapt to the valid concerns surrounding its use in food production.

Moreover, despite recent nanotechnological studies have offered several alternative nano-sized materials that are cheap, biodegradable, safe, biocompatible, and easily reproducible from a natural source, the number of biodegradable nanomaterials tested for potential use in plant agriculture is extremely limited.

Therefore, there remains a need to develop nanomaterials which are derived from natural source such that they are safe and environmental friendly for being used in plant and other agriculture products.

### SUMMARY OF THE DISCLOSURE

Certain aspects of the disclosure are directed to applications of cellulose nanocrystals (CNC) as delivery carriers for the transport of chemicals, e.g. growth regulators, pesticides, and large molecules including polynucleotides and polypeptides across plant cell walls. The large molecules include DNA e.g. CRISPR-Cas9, RNA e.g. miRNA, siRNA, proteins e.g. antibodies and enzymes, and other particles such as bacteriophages.

In one aspect of the invention, a nanomaterial conjugate for delivering an active agent to a plant, the nanomaterial conjugate comprising a nanocarrier and an active agent linked to the nanocarrier, wherein the nanocarrier is cellulose nanocrystal (CNC) and the nanocarrier delivers the active agent across cell walls of cells of the plant.

In one embodiment, the active agent comprises a growth regulator.

In one embodiment, the growth regulator comprises an agrochemical.

In one embodiment, the agrochemical is 2,4-dichlorophenoxyacetic acid (2,4-D).

In one embodiment, the active agent comprises a large molecule.

In one embodiment, the large molecule comprises a polynucleotide.

In one embodiment, the large molecule comprises a polypeptide.

In one embodiment, effective dose of the nanomaterial conjugate is lower than effective dose of the active agent alone.

In one embodiment, the nanocarrier and the active agent are covalently linked.

In one embodiment, the nanocarrier and the active agent are non-covalently linked.

In one embodiment, the nanocarrier is not toxic to the plant at a concentration below 100 g/L.

In one embodiment, the nanomaterial conjugate is not toxic to the plant at a concentration below 500 mg/L.

In another aspect of the invention, a method for delivering an active agent across cell walls of plant cells, comprising providing the plant cells in a medium; introducing an amount of a nanomaterial conjugate to the medium to form a mixture thereof; and maintaining the mixture at a temperature for a period of time to allow sufficient interaction of the plant cells with the nanomaterial conjugate, wherein the nanomaterial conjugate comprises a nanocarrier and an active agent linked to the nanocarrier; wherein the nanocarrier is cellulose natural crystal; and wherein the nanocarrier delivers the active agent across the cell walls of the plant cells.

In one embodiment, the active agent comprises a growth regulator.

In one embodiment, the growth regulator is 2,4-dichlorophenoxyacetic acid (2,4-D).

In one embodiment, the active agent comprises a large molecule.

In one embodiment, effective dose of the nanomaterial conjugate is lower than effective dose of the active agent alone.

In one embodiment, the effective dose of the nanomaterial conjugate is less than 50% of the effective dose of the active agent alone.

In one embodiment, the nanomaterial conjugate is applied at a concentration in a range of 0.1 mg/L to 500 mg/L.

### BRIEF DESCRIPTION OF THE DRAWINGS

The accompanying drawings illustrate one or more embodiments of the disclosure and together with the written description, serve to explain the principles of the disclosure. Wherever possible, the same reference numbers are used throughout the drawings to refer to the same or like elements of an embodiment.

FIG. 1A shows visualization of germination of tomato seeds sprayed with CNC in a wide range of doses (0, 10, 50, 100, 500, 1000 mg/L in upper panel and 0, 20, 40, 60, 80, 100 g/L in lower panel). FIG. 1B shows recorded germination rate of tomato seeds sprayed with CNC in a wide range of doses (0, 20, 40, 60, 80, 100 g/L). FIG. 1C shows effect of CNC added in hydroponics system (1 g/L) on the phenotype of tomato plants including the number of leaves, flowers, fruits. FIG. 1D shows effect of CNC added in hydroponics system (1 g/L) on the phenotype of tomato plants including length of stems and roots. FIG. 1E shows effect of CNC added in hydroponics system (1 g/L) on the phenotype of tomato plants in terms of the weight of total fresh plant biomass. FIG. 1F shows effect of CNC added in hydroponics system (1 g/L) on the phenotype of tomato plants the weight of total dry plant biomass. FIG. 1G shows the application method of CNC in FIGS. 1C-F.

FIG. 2A shows principal component analysis (PCA) represents significant differences in transcriptomes of green tomato fruits and leaves collected from untreated tomato plants and same organs collected tomato plants treated with 50 mg/L and 1 g/L of CNC for 3 weeks. FIG. 2B shows the metabolomes of both green and red tomato fruits. FIG. 2C shows the metabolomes of both green and red tomato leaves. FIG. 2D shows top 10 transcriptomic pathways affected in tomato green fruits (upregulated). FIG. 2E shows top 10 transcriptomic pathways affected in tomato green fruits (downregulated). FIG. 2F shows top 10 transcriptomic pathways affected in tomato green leaves (upregulated). FIG. 2G shows top 10 transcriptomic pathways affected in tomato green leaves (downregulated).

FIG. 3A shows a representative structure of sulfated CNC and image of individual feedstock CNC obtained via AFM. FIG. 3B shows a diagram of synthesis process of DTAF-labeled CNC and confirmation of attachment after extensive dialysis via fluorescent microscopy. FIG. 3C shows a diagram of synthesis process of amine functionalized CNC and continued reactions with (I) FITC and (II) 2,4-D.

FIG. 4A shows a diagram of experimental approach of using dyes DTAF and FITC for monitoring of absorption of CNC attached to dyes by Arabidopsis leaf cells and tobacco cells with cell wall (callus) and without cell wall (protoplasts). FIG. 4B shows a fluorescence intensity image of Arabidopsis leaves incubated with water. FIG. 4C shows a fluorescence intensity image of Arabidopsis leaves incubated with CNC. FIG. 4D shows a fluorescence intensity image of Arabidopsis leaves incubated with DTAF. FIG. 4E shows a fluorescence intensity image of Arabidopsis leaves incubated with CNC-DTAF conjugates. FIG. 4F shows mean fluorescent intensity (f). FIG. 4G shows a fluorescence intensity image of tobacco protoplasts treated with water. FIG. 4H shows a fluorescence intensity image of tobacco protoplasts treated with CNC. FIG. 4I shows a fluorescence intensity image of tobacco protoplasts treated with CNC-FITC conjugates. FIG. 4J shows a fluorescence intensity image of tobacco callus cells treated with water. FIG. 4K shows a fluorescence intensity image of tobacco callus cells treated with CNC. FIG. 4L shows a fluorescence intensity image of tobacco callus cells treated with CNC-FITC conjugates. FIG. 4M shows a chart comparing measured fluorescence intensity of treated cells.

FIG. 5A shows graphic assessment of growth of tobacco calluses in MS medium No. 1-6.FIG. 5B shows fresh weights of calli grown on MS medium No. 1-6. FIG. 5C shows tobacco calluses grown on MS media supplemented with either 0, 10 mg/L, 50 mg/L, or 500 mg/L CNC. FIG. 5D shows total biomass accumulation of calli grown on CNC-supplemented MS media.

### DETAILED DESCRIPTION OF THE DISCLOSURE

The disclosure will now be described more fully hereinafter with reference to the accompanying drawings, in which exemplary embodiments of the disclosure are shown. This invention may, however, be embodied in many different forms and should not be construed as limited to the embodiments set forth herein. Rather, these embodiments are provided so that this disclosure will be thorough and complete, and will fully convey the scope of the disclosure to those skilled in the art. Like reference numerals refer to like elements throughout.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the disclosure or of any exemplified term. Likewise, the disclosure is not limited to various embodiments given in this specification.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the disclosure.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top”, may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “around”, “about”, “substantially” or “approximately” shall generally mean within 20 percent, preferably within 10 percent, and more preferably within 5 percent of a given value or range. Numerical quantities given herein are approximate, meaning that the terms “around”, “about”, “substantially” or “approximately” can be inferred if not expressly stated.

As used herein, the terms “comprise” or “comprising”, “include” or “including”, “carry” or “carrying”, “has/have” or “having”, “contain” or “containing”, “involve” or “involving” and the like are to be understood to be open-ended, i.e., to mean including but not limited to.

As used herein, the phrase “at least one of A, B, and C” should be construed to mean a logical (A or B or C), using a non-exclusive logical OR. It should be understood that one or more steps within a method may be executed in different order (or concurrently) without altering the principles of the disclosure.

As used herein, the term, “nanosized material”, refers to an object of intermediate size between molecular and microscopic (micrometer-sized) materials. In describing nano-sized materials, the sizes of the nano-sized materials refer to the number of dimensions on the nanoscale. A list of nano-sized materials includes, but are not limited to, nanoparticle, nanocomposite, quantum dot, nanofilm, nanoshell, nanofiber, nanowire, nanotree, nanobush, nanotube, nanoring, nanorod, and etc.

As used herein, the term “effective dose” refers to dose of a composition which, when administered to a subject, e.g a plant or a part of the plant, is effective to cause a desired effect resulted by the composition.

The description below is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the disclosure.

Cellulose nanocrystals (CNC) are nanoscale components of all cellulosic biomass. They can also be extracted from tunicates and produced by bacteria. CNC are often produced by the sulfuric acid hydrolysis of wood biomass resources. The resulting structure has a length on the order of 100 nm and a diameter on the order of 5 nm. CNC are often considered as rod-like, but their actual cross section is not circular and the third dimension is often on the order of tens of nm. CNC are great materials for nanocarrier design due to their large aspect ratio, high specific surface area, and renewable resource origin. Recently, CNC have been utilized in water treatments to remove carcinogenic dyes, sprays to protect fruit tree blossoms from frost damage. Biological polysaccharides like CNC are appealing tools for agricultural applications due to their lower risk of toxicity and numerous hydroxyl groups to target for surface modification. The surface chemistry of CNC provides flexibility when chemically grafting compounds on their surface. These chemical characteristics make CNC favorable for use in conjugation with many of the agents widely used in agriculture.

Agrochemicals such as growth regulators, herbicides, antifungal treatments, and other compounds are commonly applied through foliar sprays and treatments. However, these existing procedures have significant limitations. It is understood that only a fraction (sometimes as low as 0.1%) of the applied agent will reach its target due to rain wash-off, UV degradation, wind drift, and volatilization.

The present invention has developed technologies of using CNCs as delivery carriers for the transport of chemicals such as growth regulators and pesticides and nucleic acids such as DNAs and RNAs through a plant cell wall.

Moreover, the present invention discloses schemes for attachment of growth regulators or large molecules to the surface of CNC and demonstrates uptake of CNC conjugates by plant cells.

The present invention discloses using of CNC as a nanocarrier system for the delivery of agrochemicals, which promotes the absorption of applied agrochemicals. In one embodiment, the present invention reduces the effective dose of agrochemical needed for the desired effect. In one embodiment, the present invention evaluates the ability of CNC to penetrate the plant cell wall and deliver the model growth regulator 2,4-D to plant cells.

Nanosized materials can possess novel properties and affect exposed organisms in unexpected ways. For example, previously it has been reported that nanoscale carbon-based materials can cause biological effects in planta that were not observed with activated carbon. Multi-walled carbon nanotubes (MWCNT) introduced in a growth medium in low doses enhanced the growth of tobacco cell culture, but activated carbon used in the same concentration did not cause this effect. Similarly, MWCNT, but not activated carbon, stimulated the production of flowers and fruits in exposed tomato plants.

However, comparing to other nanomaterials, CNC generally have larger diameter, particularly in their larger cross-section width, as well as unique surface chemistry, and thus may demonstrate its unique properties comparing to other nanomaterials e.g. CNT, when being used applied to plants, e.g. assisting delivery of active agents across the plant cell walls.

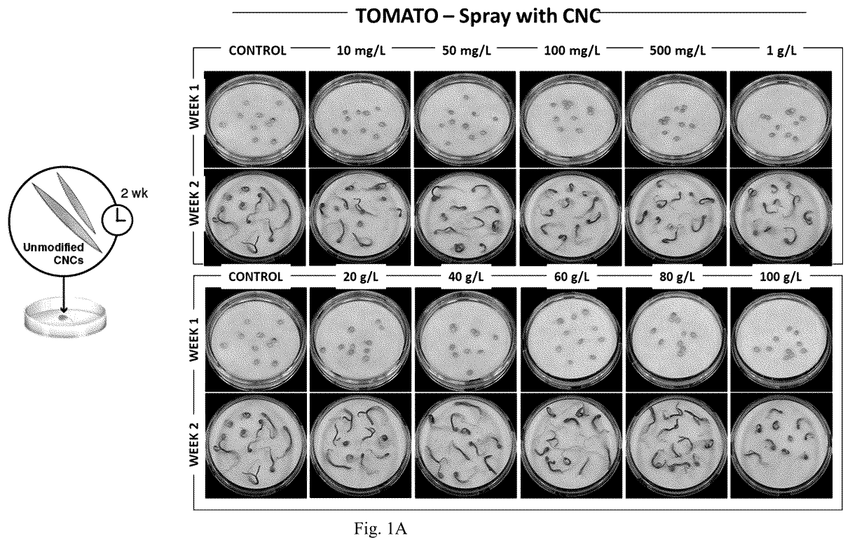

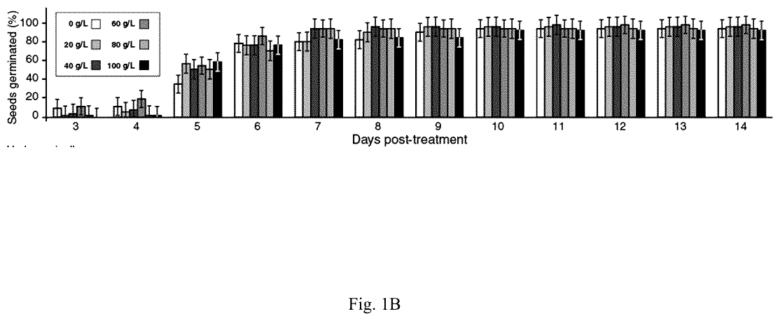

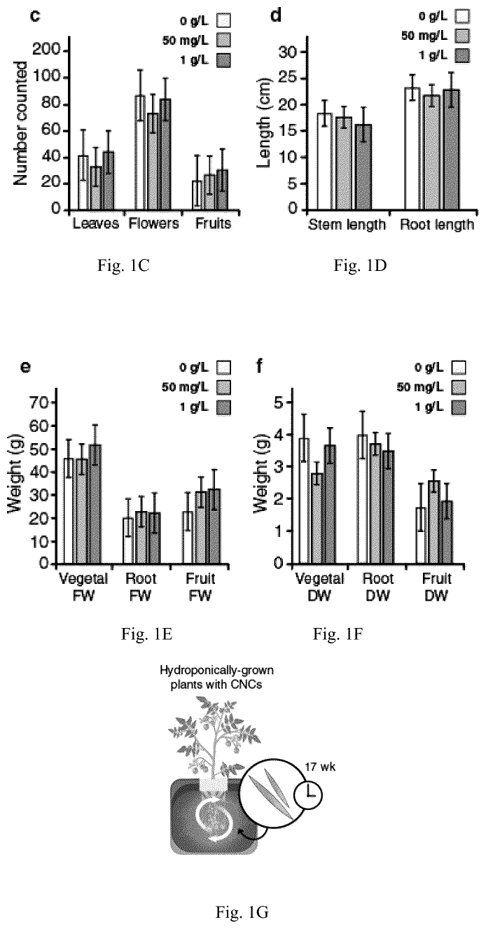

Therefore, before CNC can be safely applied to plants, it was necessary to comprehensively characterize the impact of the material on plants at a physiological and molecular level. Here, the present invention characterize the response of tomato seeds and mature plants to the application of commercially-available sulfated CNC derived from woody biomass as shown in FIGS. 1A-F and 2A-G.

FIGS. 1A-G show assessment of potential phytotoxicity caused by CNC applied to tomato seeds and tomato plants grown in a hydroponics system. FIG. 1A shows the visualization of germination of tomato seeds sprayed with CNC in a wide range of doses (0, 20, 40, 60, 80, 100 g/L) throughout 14 days. FIG. 1B show the recorded germination rate of tomato seeds sprayed with CNC in a wide range of doses (0, 10, 50, 100, 500, 1000 mg/L in upper panel and 0, 20, 40, 60, 80, 100 g/L in lower panel). FIGS. 1C-F shows effects of CNC added in hydroponics system (1 g/L) on the phenotype of tomato plants. In particular, FIG. 1G shows the application method of CNC to the plant. FIG. 1C shows the number of leaves, flowers, fruits; while FIG. 1D shows length of stems and roots. FIG. 1E shows the weight of total fresh plant biomass, and FIG. 1F shows the weight of total dry plant biomass. Statistical analysis of phenotypical measurements determined with one-way ANOVA, α=0.05. All error bars represent standard error (SE).

As shown in FIGS. 1A-B in performed germination tests, CNC sprayed in relatively low doses (0, 10, 50, 100, 500, 1000 mg/L) and in high doses (0, 20, 40, 60, 80, 100 g/L) did not affect the germination of exposed tomato seeds. Similarly, tomato plants treated with CNC (17 weeks of CNC exposure through hydroponics) did not exhibit any significant changes in major phenotypical plant traits such as plant height, root length, number of leaves, fruits, or branches, overall biomass produced, total leaf area, or regarding photosynthetic performance in CNC-exposed tomatoes, according to FIGS. 1C-F.

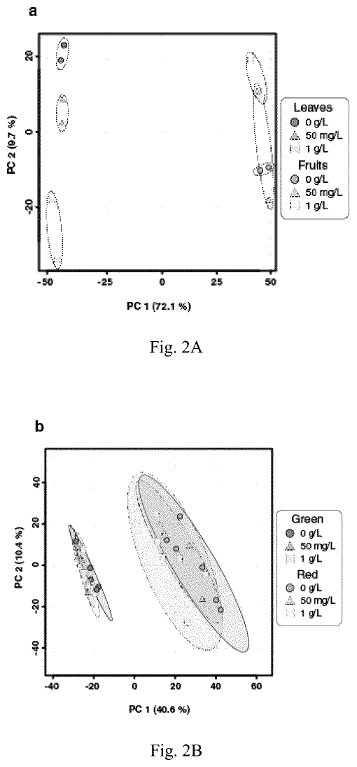

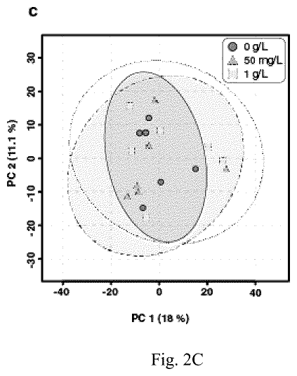

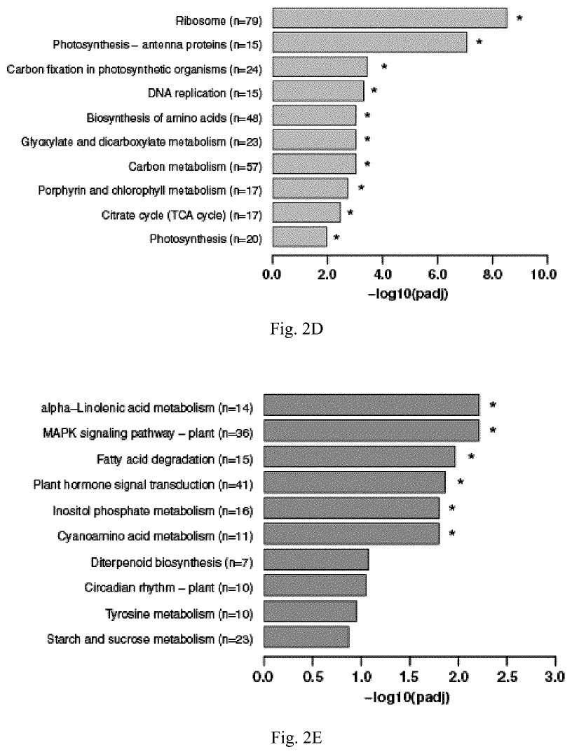

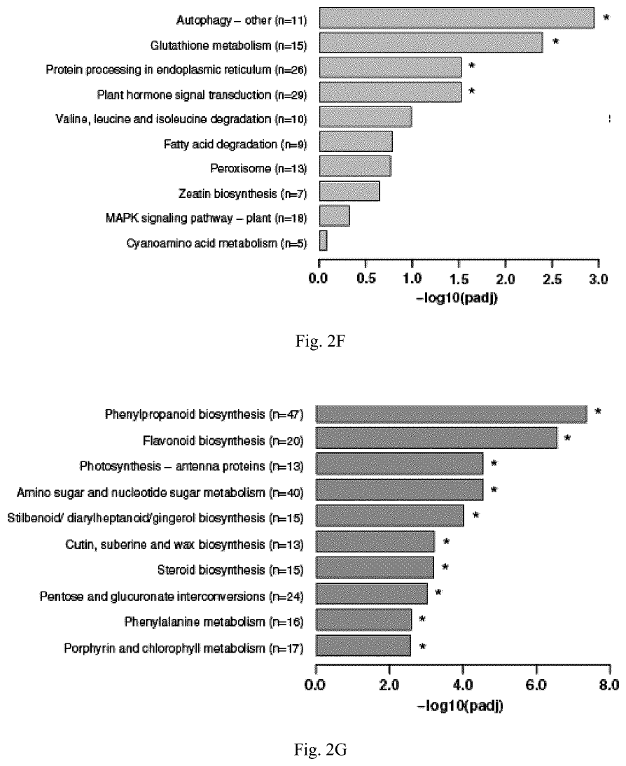

To understand how applied CNC can impact plants at a molecular level, the total transcriptome and total metabolome of CNC-exposed tomatoes was investigated using RNA-seq and LC-MS methods. FIGS. 2A-G show effects of CNC in total transcriptomes and metabolomes of tomato plants grown in presence of CNC (1 g/L) added to hydroponics system.

The present invention discovers that long-term CNC treatments through hydroponics (50 mg/L; 1 g/L) significantly affected the total gene expression in fruits and leaves of CNC-exposed tomato plants, according to FIG. 2A, and was especially noticeable for genes involved in pathways related to photosynthesis, signal transduction, secondary metabolism, and both starch and sugar metabolism.

In one embodiment, CNC treatment can also be applied to plants via root/leaf uptake, or other application methods commonly used in agriculture.

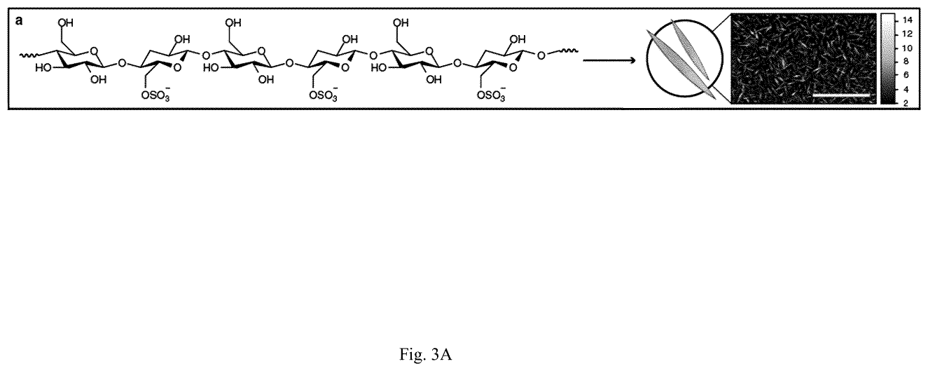

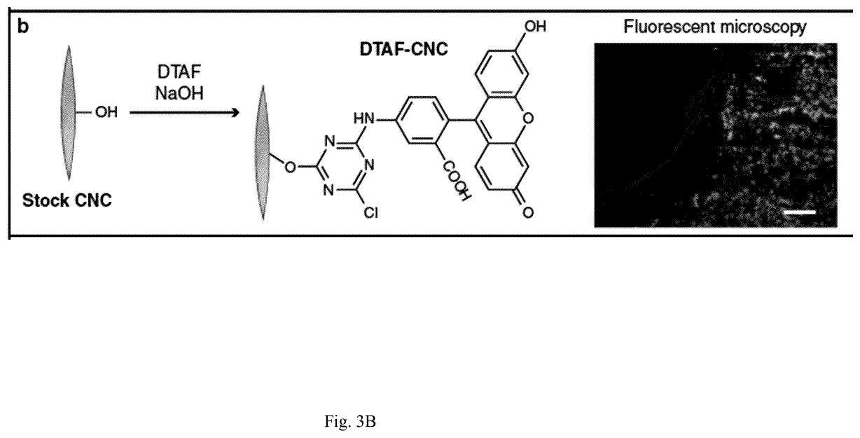

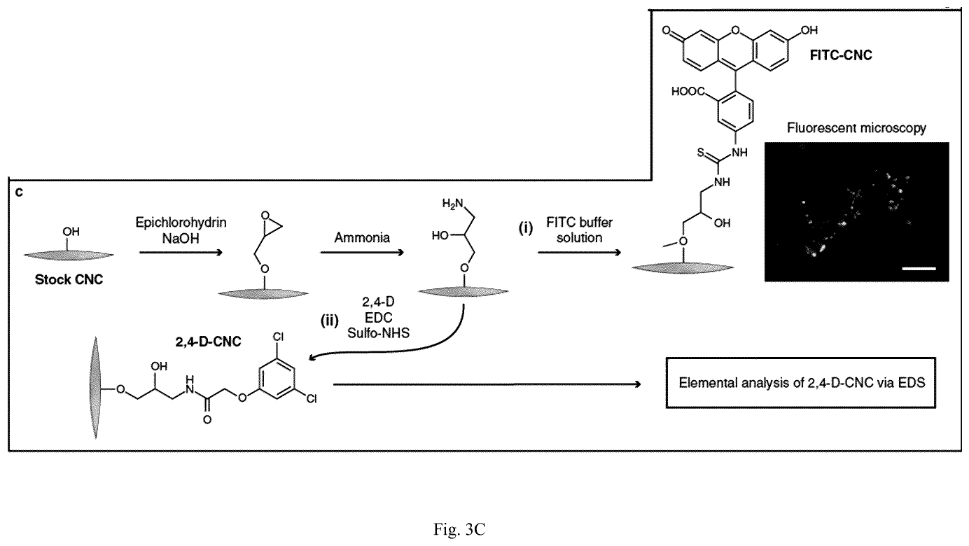

FIG. 2A shows that principal component analysis (PCA) represents significant differences between transcriptomes of green tomato fruits and leaves collected from untreated tomato plants and same organs collected tomato plants treated with 50 mg/L and 1 g/L of CNC for 3 weeks. FIGS. 2B-C shows that PCA represents lack of significant differences between metabolomes of both green and red tomato fruits (FIG. 2B) and (FIG. 2C) leaves collected from untreated tomato plants and same organs collected tomato plants treated with 50 mg/L and 1 g/L of CNC for 4 weeks. Each dot represents an analyzed individual sample in FIGS. 2A-C. Surrounding clouds in FIG. 2B and FIG. 3C are related to 95% confidence intervals.

FIGS. 2D-G show top 10 transcriptomic pathways affected in tomato green fruits (FIG. 2D and 2E) and leaves (FIG. 2F and 2G) by 1 g/L CNC exposure for 3 weeks. Pathways with upregulated (orange) and downregulated (blue) compounds were identified by comparison of the organ's list of upregulated or downregulated metabolites to the KEGG database for Solanum lycopersicum. Numbers (n=) the end of each bar indicate the number of transcripts associated with the pathway that were dysregulated comparing to untreated tomato organs. Asterisks denote significantly affected pathways, p=<0.05.

However, at the metabolic level, statistically significant changes in total metabolome of fruits and leaves of CNC-treated plants were not detected as evident in PCA, according to FIG. 2B and 2C. A lack of detectable phytotoxicity caused by CNC at physiological and molecular levels provides evidence that CNC are safe for use with plants. Previously, the ability of CNC and CNC-conjugates to enter animal cells was investigated with favorable results, demonstrating the possibility for using nanocellulose for drug delivery into implants or target cells. Liebert et al. described significant uptake of FITC-labeled cellulose nanospheres by human fibroblasts. Sulfated CNC labeled with RBITC and FITC fluorophores and found that cationic, amino-terminated CNC-RBITC conjugate could penetrate human kidney cells (HEK 293) and the cells of Spodoptera frugiperda (SD) without detectable toxicity. The transport of biomolecules and chemicals to plant cell is known to be more challenging compared to animal cells due the presence of a rigid cell wall. To explore the possibility of delivering fluorescent dyes and growth regulators to plant cells using CNC nanocarriers, CNC were conjugated with two types of dyes (DTAF, FITC) and widely used agrochemical 2,4-dichlorophenoxyacetic acid (2,4-D). Conjugation and confirmation of CNC-conjugate attachment

Before conjugation with fluorescent dyes and 2,4-D, the CNC stock was first characterized with atomic force microscopy (AFM) to determine their shape and size. According to FIG. 3A, on average, stock CNC were 160 nm in length 3 nm in height with a width of 60 nm as measured by atomic force microscopy. Since the CNC stock was derived from sulfuric acid hydrolysis, some of the native surface hydroxyl groups were replaced with sulfate half esters. Using the method of Abitol et al., the stock had a sulfur content of 0.6%. The characterization of stock CNC is shown in FIG. 3A. In one embodiment, CNC of different dimensions are used for conjugation without deviating from the present invention.

Reaction pathway used for the synthesis of DTAF, FITC, and 2,4-D conjugated CNC is represented in FIGS. 3B-C.

As shown in FIG. 3B, CNC-DTAF conjugation was completed in a one-step synthesis under alkaline conditions, whereas the CNC-FITC and CNC-2,4-D conjugates required the formation of a CNC-NH2 intermediate to enable conjugation with the target compounds. After amine functionalization, CNC-FITC conjugation was achieved by incubating the functionalized CNC with FITC buffer solution, and CNC-2,4-D conjugation was achieved by utilizing (N-(3-dimethylaminopropyl)-N′-ethylcarbodiimide hydrochloride (EDC) and N-hydroxysulfosuccinimide sodium salt (NETS) chemistry as described by the schema in FIG. 3C. Before established CNC conjugates were delivered to leaves, protoplasts, and cells, the successful conjugation of fluorescent dyes and 2,4-D to CNC was confirmed. DTAF and FITC attachment to CNC was confirmed via fluorescent microscopy after extensive purification of the conjugates. Quantification of primary amine and 2,4-D functionalization of CNC was determined through CHNS elemental analysis and energy dispersive x-ray spectroscopy (EDS), respectively. Feedstock sulfated CNC does not contain nitrogen so the amount of nitrogen measured by EDS was used to determine the fraction of hydroxyl groups functionalized with amines in the initial reaction step. Elemental analysis of chlorine content in the final product was used to quantify the amount of 2,4-D in the 2,4D-CNC conjugate since chlorine is only present in 2,4-D. The weight percentage of chlorine was estimated using EDS, the most common method in literature to quantify CNC surface modification. Changes in reaction conditions were used to produce 2,4D-CNC conjugates containing from less than 0.1% to over 0.4% 2,4D by mass.

### Delivery of Fluorescent Dyes to Leaves, Protoplasts and Callus Cells

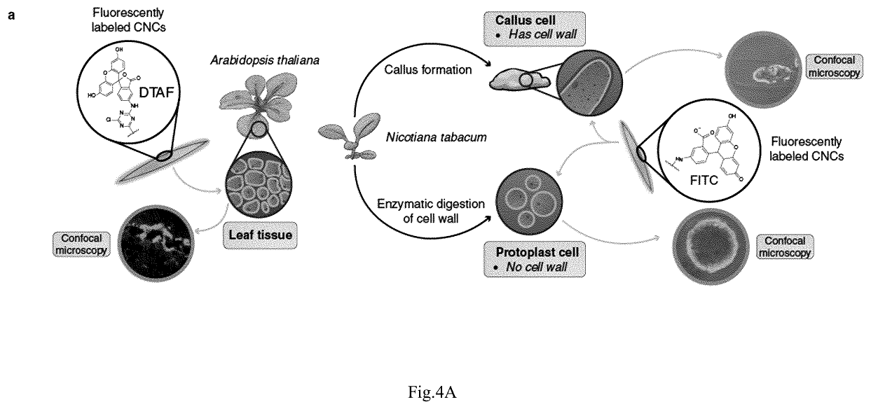

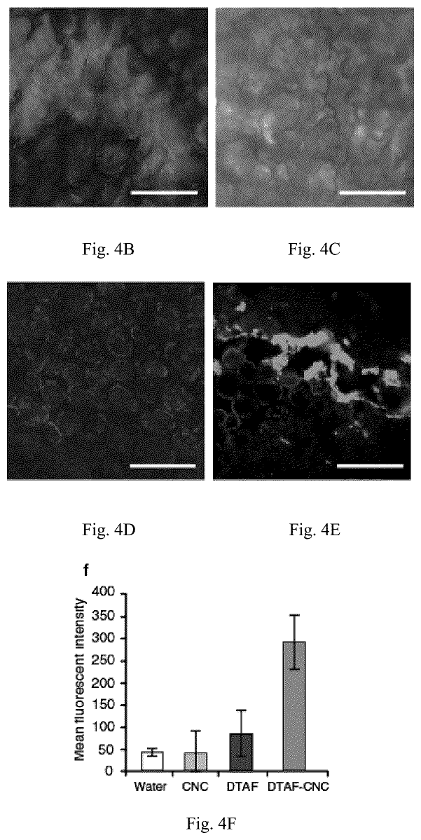

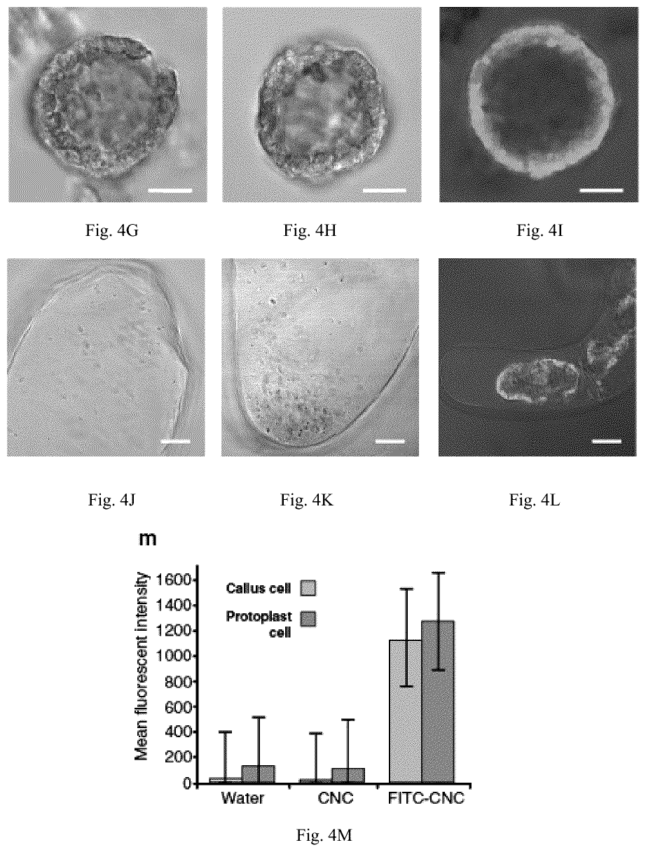

To evaluate entry of synthesized CNC-dye conjugates into leaves, 12-day-old Arabidopsis thaliana seedlings were soaked with CNC-DTAF conjugate solution for 4 hours. Additionally, both protoplasts and callus cells of Nicotiana tabacum were incubated with CNC-FITC conjugate for 4 hours, as shown in FIG. 4A. All samples were imaged with confocal microscopy. The fluorescence of CNC-DTAF and CNC-FITC stained samples was digitally measured to determine the difference in signal intensity between conjugate-treated and untreated plant samples.

FIGS. 4B-F show fluorescence intensity images and measured fluorescence intensity (f) of Arabidopsis leaves incubated with water (FIG. 4B), CNC (FIG. 4C), DTAF (FIG. 4D) and CNC-DTAF conjugates (FIG. 4E), for 4 hours.

As shown in FIG. 4B, only slight autofluorescence was observed for untreated A. thaliana leaves and leaves treated only with free CNC or DTAF in doses comparable with amounts of CNC and dye used in CNC-DTAF conjugate (negative controls), as shown in FIGS. 4C-E. Incubation of A. thaliana leaves with CNC-DTAF conjugate solution resulted in an 82% increase in mean fluorescence intensity compared to water treated samples and 76% increase compared to free DTAF treatment, as shown in FIG. 4F. This observation indicated the ability of CNC to penetrate the cell wall and deliver dye inside the cell.

To further confirm entry of CNC in plant cells, in one embodiment, the present invention incubated both N. tabacum callus cells (possessing cell walls) and protoplasts (lacking cell walls) with either CNC-FITC dye conjugates or pure CNC to measure the level of fluorescence, as shown in FIGS. 4G-M. A dramatic increase of fluorescence was noted after 4 hours of incubation with CNC-FITC conjugates in both callus and protoplast cells (99% and 90% increased respectively). In particular, FIG. 4G-L show fluorescence intensity images of tobacco protoplasts. FIG. 4G shows a fluorescence intensity image of tobacco protoplasts treated with water for 4 hours. FIG. 4H shows a fluorescence intensity image of tobacco protoplasts treated with CNC for 4 hours. FIG. 4I shows a fluorescence intensity image of tobacco protoplasts treated with CNC-FITC conjugates for 4 hours. FIG. 4J shows a fluorescence intensity image of tobacco callus cells treated with water for 4 hours. FIG. 4K shows a fluorescence intensity image of tobacco callus cells treated with CNC for 4 hours. FIG. 4L shows a fluorescence intensity image of tobacco callus cells treated with CNC-FITC conjugates for 4 hours. FIG. 4M shows a chart comparing measured fluorescence intensity of treated cells. Scale bars represent 50 μm for leaf in FIGS. 4B-E, 10 μm for protoplasts for FIGS. G-I and 20 μm for callus cells for FIGS. J-L. Error bars represent SE, asterisks denote significant values, p=<0.05.

### Delivery of Growth Regulator 2,4-D to tobacco cells

The multistep scheme illustrated in FIG. 3C resulted in CNC-2,4-D conjugates which were 0.05% 2,4-D by mass. To understand if CNC can deliver a plant hormone that encourages cell division to plants, CNCs conjugated with 2,4-D were added to MS medium. 2,4-D was selected for conjugation based on the extensive use of this synthetic auxin as both a plant growth regulator commonly employed in tissue cultures and as an active ingredient in commercial herbicides. At a modest dose of 1 mg/L, 2,4-D can act as a positive growth regulator and is commonly used in plant cell cultures. At the same time, higher doses of 2,4-D can exhibit herbicidal activity, leading to it being one of the most extensively used herbicides for industrial and commercial use, and considered a model agrochemical for laboratory and field testing. To estimate if the conjugation of CNC and 2,4-D can improve the uptake of 2,4-D, the total biomass of callus tissues cultivated on media containing 2,4-D was measured. N. tabacum calli were seeded on media containing either a standard dose of 2,4-D (1 mg/L) or a reduced dose of 2,4-D (0.4 mg/L) both conjugated and non-conjugated with CNC.

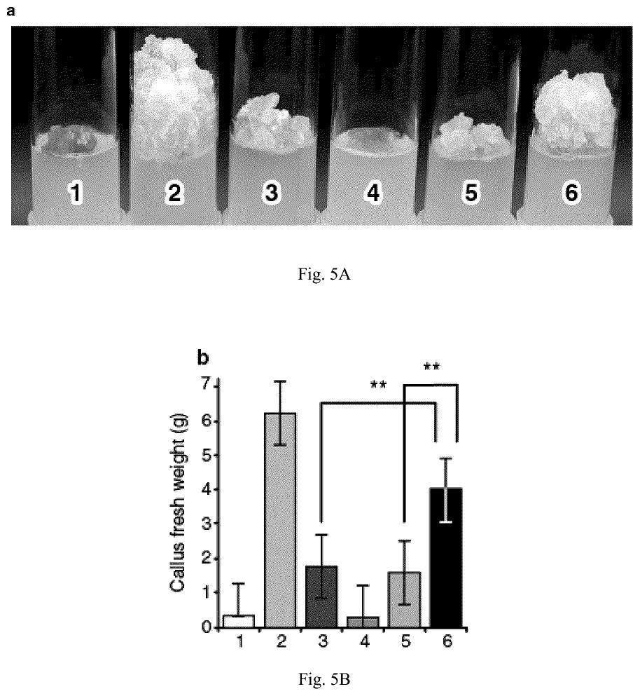

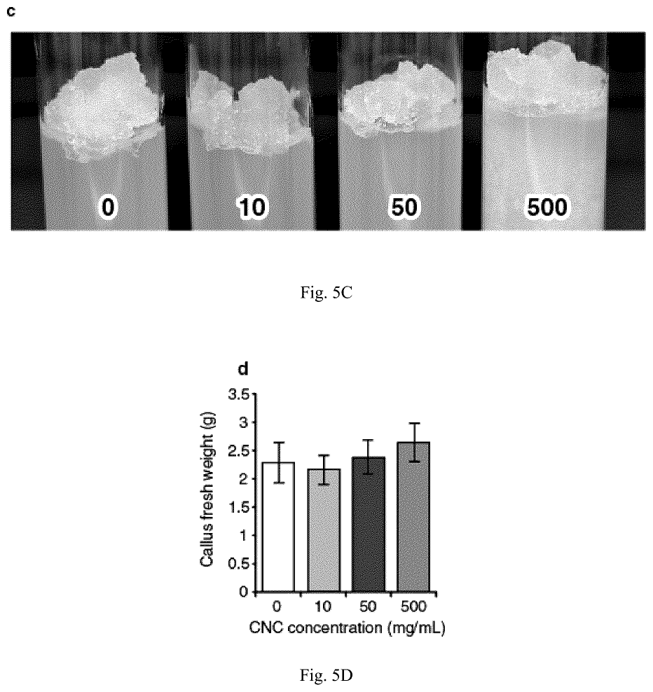

Tobacco callus cells cultivated on media supplemented with CNC or CNC-2,4-D conjugates. FIG. 5A shows graphic assessment of growth of tobacco calluses in MS medium without 2,4-D addition (medium 1), in MS medium supplemented with standard concentration of 2,4D (1 mg/L) (medium 2), medium with reduced concentration of 2,4-D (0.4 mg/L) (medium 3), medium without 2,4-D but supplemented with 0.4 mg/L CNC (medium 4), medium supplemented with non-attached 2,4-D (0.4 mg/L) and CNC (0.4 mg/L) (medium 5), and medium supplemented with 2,4-D-CNC conjugate (equivalent to 0.4 mg/L 2,4D). The tubes containing mediums 1-6 and tobacco callus are presented at the end of cultivation period (35 days). FIG. 5B shows comparison chart of fresh weights of calli grown on medias 1-6 for 35 days. Asterisks denote significantly different factors determined by t-test, p=<0.05.

FIG. 5C shows tobacco calluses grown on MS media supplemented with either 0, 10 mg/L, 50 mg/L, or 500 mg/L CNC. FIG. 5D shows comparison chart of total biomass accumulation of calli grown on CNC-supplemented MS media. No significant differences in final callus weight were detected by one-way ANOVA. All error bars represent SE.

MS mediums without 2,4-D and without 2,4-D but supplemented with CNC in similar concentrations to the CNC-2,4-D conjugate were used as negative controls. CNC alone were determined to not have a significant effect on callus growth, according to FIGS. 5C-D. After 35 days of cultivation in the dark, the total fresh weight of the cell culture was estimated for all growth mediums. As expected, no growth of tobacco calluses was observed on mediums not supplemented with 2,4-D or supplemented only with CNC, according to FIG. 5B. As expected, suppressed cell growth was observed in medium supplemented with a reduced amount of 2,4-D (0.4 mg/L) with or without the addition of CNC compared to the growth of calli cultivated on medium supplemented with a regular dose of 2,4-D (1 mg/L).

However, when the CNC-2,4-D conjugate was added to the medium as a source of 2,4-D at a reduced rate (0.4 mg/L), the growth of cell culture was only moderately reduced in comparison to the medium with regular 1 mg/L of 2,4-D. Thus, combined with confocal microscopy images of FIGS. 4B-E and 4G-I, the present invention shows the potential of CNC as a carrier of agricultural chemicals.

In one embodiment, the effective dose of CNC-2,4-D conjugate is 80% of the effective dose of 2,4-D alone without CNC. In one embodiment, the effective dose of CNC-2,4-D conjugate is 70% of the effective dose of 2,4-D alone without CNC. In one embodiment, the effective dose of CNC-2,4-D conjugate is 60% of the effective dose of 2,4-D alone without CNC. In one embodiment, the effective dose of CNC-2,4-D conjugate is 50% of the effective dose of 2,4-D alone without CNC. In one embodiment, the effective dose of CNC-2,4-D conjugate is 40% of the effective dose of 2,4-D alone without CNC. In one embodiment, the effective dose of CNC-2,4-D conjugate is 30% of the effective dose of 2,4-D alone without CNC. In one embodiment, the effective dose of CNC-2,4-D conjugate is 20% of the effective dose of 2,4-D alone without CNC.

In addition, the present invention also proves that pure CNC in a wide range of doses (1, 50, 500 mg/L) are not toxic for tobacco cell culture and do not result in a reduction of biomass at the end of cultivation period, according to FIGS. 5C-D.

### Conclusions

The present invention, for the first time, has demonstrated the ability of CNC, a biocompatible and sustainable biopolymer, to enter plant cells and deliver chemicals (dyes, growth regulators) to leaves and plant cells. The effect of model agrochemical and herbicide 2,4-D can be significantly enhanced by attachment to CNC. The present invention has also provided experimental evidence that a high-dose application of CNC to seeds and plant organs does not lead to phytotoxicity, genotoxicity, or other undesirable effects in plants. The present invention shows that CNC is a safe and environmentally friendly nanomaterial and can be used as nanocarriers for important active agents with less concern for bioaccumulation and later issues with toxicity. Conjugation of bioactive compounds with CNC has the potential to increase the efficacy of bioactive compounds such as 2,4-D by acting as an entry vehicle into plant cells, which paves the way for many different applications. Depending on the context, the increased bioavailability of attached compounds may allow for a reduction in the volume of active agent to achieve a desired result. In an agricultural context, using CNC-based conjugates for other common herbicides offers more potent effects than bare agent alone. This is quite beneficial to the agricultural industry for several reasons. One such example is curtailment of herbicide-resistance in weedy plants. Herbicide resistance is effectively positively selected for when weeds survive through a herbicidal treatment. While herbicide resistance will continue to be an issue, more effective and potent formulations of existing herbicides may reduce the risk of resistance spreading.

Another beneficial aspect of CNC-agrochemical conjugation is the modification of undesirable chemical properties, such as volatilization. Some agrochemicals such as 2,4-D are known to be quite volatile, and may evaporate after application if conditions are hot and dry enough. This can result in less herbicide remaining to act upon the target weeds, and issues with wind drift and off-target damage to nearby, susceptible crops. As shown by the present invention, covalent conjugation of an active agent to solid CNC could reduce volatilization. The tunable surface chemistry of CNC provides a suitable canvas for the attachment of many different compounds, including protein attachment. The present invention of CNC nanocarriers penetrating the cell wall and delivering bioactive compounds into plant cells will promote innovative platforms for the applications of nanocellulose particles in plant genetic engineering and agriculture.

### Materials and Methods

### Characterization of Stock CNC

### Physical Characterization

Acid hydrolyzed CNC stock was purchased from CelluForce Inc. (Montreal, Canada). Atomic force microscopy was used to characterize the physical properties of the stock CNC. The zeta potential of 2500 ppm aqueous CNC was measured using a Malvern Zetasizer Nano ZS (Malvern, UK).

### Thermogravimetrics

For thermogravimetric analysis, a TA Instrument TGA Q50 (New Castle, DE) was used under inert gas flow. After loading the CNC, the sample was heated at 10° C./min to 120° C. and then held isothermal for 30 minutes to remove all residual moisture. CNC was then ramped at 5° C./min to 800° C. and held isothermal for 30 minutes before cooling. The thermal degradation temperature was calculated based on 5% mass loss of CNC after the isothermal hold at 120° C.

### Charge Density

The charge density of CNC was determined by colloidal titration. The CNC was diluted to 0.5 wt % in ultra-pure water, and 25 mL of p-DADMAC (0.001 N) was added to 15 mL dispersion. The mixture was centrifuged to separate any aggregates. 10 mL supernatant was collected and titrated against 0.001 N PVSK using a Chemtrac Laboratory Charge Analyzer (Fremont, CA).

### Sulfate Half Ester Determination

Determination of sulfur content of the stock CNC was based on the presence of sulfate half ester groups. CNC was diluted to 1.0 wt % and then dialyzed extensively (Spectra/Por membrane, 12 kDa cutoff) with ultra-pure water for five days until the pH of the dialyzed water became equal to the pH value of pure water. The dialyzed dispersion was further diluted to 0.5 wt % with ultra-pure water. An ion exchange column was prepared with 40 g of DOWEX Marathon-C resin and washed with ultra-pure water until the pH and color of the rinsed water became same as ultrapure water. The 0.5 wt % CNC dispersion was passed through the column three times. Then, dispersion containing 150 mg of the protonated CNC was diluted to reach a volume of 200 mL. To increase the initial conductivity of the CNC, 2 mL of 0.1 M NaCl solution was added. Then the obtained suspensions were titrated with 10 mM NaOH using the titrator system Symphony B30PCI to determine the equivalence point).

### Biological Assessments of CNC and Tomato Plants

### CNC Treatment of Tomato Seeds

Tomato seeds (cv. Micro-Tom) were sterilized as previously described. 50 seeds were used for each CNC concentration (10, 50, 100, and 500 mg/L, and 1, 20, 40, 60, 80, and 100 g/L). Stock CNC was diluted with double-distilled water (ddH₂O) and sonicated to homogeneity before use. Seeds were placed on sterile dishes containing filter paper pre-wet with 2 mL of sterile ddH₂O and sprayed with CNC solutions or water (control) using airbrush as described by Lahiani et al. The dishes with treated seeds and untreated seeds (control) were sealed and incubated in a growth chamber under 12 h light (25° C.) and 12 h dark (20° C.) conditions. Germination progress was recorded daily for two weeks.

### Exposure of Tomato Plants to CNC Using a Hydroponics System

Three-week-old tomato seedlings (six plants per treatment) were transferred to 20 L Hydrofarm hydroponics units (Hydrofarm, Petaluma, CA) containing Florallova Grow Solution constantly mixed by pumps. After one week of hydroponic cultivation plants were treated with CNC over period of three weeks (CNC added once a week) to reach final concentrations of 50 mg/L or 1 g/L of CNC. Tomato tissue samples (green fruits, leaves) used for RNAseq analysis were taken after 3 weeks of tomato cultivation with CNC after reaching the desired final concentration. Samples (red/green fruits, leaves) used for LC-MS analysis were taken after 4 weeks of cultivation in presence of CNC.

### Analysis Photosynthetic Ability of Tomato Plants Exposed to CNC

Analysis of photosynthetic performance of hydroponically grown plants was conducted using a LI-6400/XT Portable Photosynthesis System (LI-COR Biosciences, Lincoln, NE) through spot analysis of mature tomato leaves in 16-week-old (13 weeks treated) plants. In a controlled setting (60% humidity, 400 html CO 2, 2000 μmol m⁻² s⁻¹ light intensity) leaves were allowed to adjust to the light for 20 minutes before taking measurements. Statistical analysis of all measured parameters was completed using SPSS software via one-way ANOVA, α=0.05.

### Analysis of Phenotype Tomato Plants Exposed to CNC

After 17-weeks of cultivation after reaching the final concentration of CNC, 23-week-old tomato plants were harvested for phenotypical analysis. The impact of CNC was assessed through several parameters: number of leaves, flowers, branches, and fruits; length of shoots and roots; weight of fruits, vegetal and root biomasses; and total leaf area for 6 control plants and 6 plants treated with each CNC dose (50 mg/L and 1 g/L). Leaf area was determined using ImageJ software. Statistical analysis of all measured parameters was completed using SPSS software via one-way ANOVA, α=0.05.

### Analysis of Transcriptome in Tomato Plants Exposed to CNC

RNA extracted from green tomato fruits and leaves were initially manually crushed and powdered using sterile mortars and pestles and liquid nitrogen. Further processing was done following the QiAGEN RNeasy Plant Mini Kit protocol (Qiagen, Redwood City, CA). The concentration and quality of isolated RNA in all samples were assessed using NanoDrop One (ThermoFisher Scientific). RNA samples were sent to Novogene Inc. (Novogene, Sacramento CA) for RNAseq analysis using the Illumina platform. Novogene provided data that included quality control and full bioinformatics analysis. Generated RNAseq data were confirmed by qPCR analysis of expression of three tomato genes: TIP2-1, HSP17.4, and P450 (CYP85A3). The 17S (18S) gene was used as internal control.

### Analysis of Metabolome in Tomato Plants Exposed to CNC

Red and green tomato fruits and leaves were collected from 8-week-old CNC-exposed (4 weeks of treatment after reaching the final concentration) tomato plants and immediately flash-frozen in liquid nitrogen. Extraction of tomato metabolites from fruits and leaves and LC-MS instrumentation was performed as described by Rezaei Cherati et al. MZmine3 software (mzmine.github.io) was used for raw data processing as was demonstrated by Du et al. MZmine3 provided initial peak alignment, gap-filling, and annotation. Annotation was based on the KEGG online database. Post-alignment data processing was conducted using Metaboanalyst to provide statistical analysis and informative pathway enrichment For all statistical measurements, any p values lower than p=0.05 were considered significant.

### Synthesis and Confirmation of CNC conjugates

### Conjugation of CNC with Fluorescent Dyes and 2,4-D

Commercial sulfatedcellulose nanocrystals in an aqueous gel (6.71 wt %) were purchased from CelluForce (Montreal, Quebec, CA). 5-[(4,6-Dichlorotriazin-2-yl)amino]fluorescein hydrochloride (DTAF), concentration ≥90% (HPLC) and was purchased from Sigma Aldrich. Fluorescein diacetate 5(6)-isothiocyanate (FITC), concentration ≥97% (HPLC) was purchased from Chemodex (Carlsbad, CA, USA). Epichlorohydrin, 2,4-dichlorophenoxyacetic acid (2,4-D), 1-ethyl-3-(3-dimethylaminopropyl)carbodiimide (EDC), and N-hydroxysulfosuccinimide (sulfo-NHS) were purchased from TCI Chemicals (Portland, OR, USA). Aqueous ammonia (28-30% ACS grade) and phosphate-buffered saline (PBS) were purchased from VWR Chemicals (Radnor, PA, USA). Sodium hydroxide was purchased from Amresco (Solon, OH, USA). All materials were used as received.

### (i) Production of DTAF Functionalized CNC

Sulfated CNCs were fluorescently tagged with DTAF in a one-step reaction under alkaline conditions. A uniform CNC dispersion (3.5 wt %) was prepared by adding water to feedstock CNC. NaOH was added to the CNC dispersion until a 0.2 M alkaline concentration was reached. DTAF was added to the alkaline CNC dispersion to maintain a CNC:DTAF mass ratio of 500:7. The mixture was stirred under a dark environment for 24 hours. The CNC-DTAF conjugate was separated from unreacted DTAF and NaOH using centrifugation (Beckman Coulter Allegra 64R, 3300 g, 30 min, six cycles) and dialysis (Spectra/Por membrane, 12 kDa cutoff). Specimen went through dialysis with ultrapure water until no trace of DTAF was found in reservoir water by UV-vis spectroscopy.

### (ii) Production of CNC-NH2

Amine functionalization was carried out using a modified method described by Dong et al. where the initial CNC concentration in an aqueous solution was 1-2% by weight.

### (iii) Production of FITC Functionalized CNC

FITC labeling was carried out in a manner described by Dong et al. Using a 1 wt % aqueous CNC-NH2 solution, FITC was added in the presence of a buffer solution until a mass ratio of 50:1 CNC:FITC was established. The reaction proceeded overnight in the dark. The conjugate underwent dialysis (Spectra/Por membrane, 12 kDa cutoff) until no FITC was detected in the reservoir water by UV-vis. Confirmation of fluorescence was conducted using a Nikon Eclipse Ti microscope equipped with an Andor Luca S camera. Images obtained were analyzed using ImageJ (NIH, version 1.48q).

### (iv) Production of 2,4-D Functionalized CNC

Molar equivalents (0.001 mols) of 2,4-D, EDC, and NHS were added under stirring to 80 mL of phosphate-buffered saline (PBS). To this, 60 g of 1 wt % aqueous CNC-NH2 was added. The reaction was then allowed to proceed for 40 hours at room temperature under stirring in a sealed flask. The product then underwent dialysis (Spectra/Por membrane, 12 kDa cutoff), tip sonication (Sonics Vibracell, 918 J/g dispersion), and second dialysis until no trace of 2,4-D was found in reservoir water by UV-vis spectroscopy.

### Degree of Surface Substitution of 2,4-D Functionalized CNC

Prior to elemental analysis all samples were oven dried overnight at 80° C. under vacuum. Samples were then run on an Elementar Vario MICRO analyzer (Ronkonkoma, NY) following the outline of ASTM D5373-02.

### Biological Assessments of CNC, CNC Conjugates, and Plant Cells

### Preparation of Tobacco Protoplasts

Nicotiana tabacum protoplasts were isolated from 4-week-old plants using a digestion solution composed of 1.5% Onozuka R-10, 0.3% macerozyme maceroenzyme R-10, 0.4 M mannitol, 20 mM KCl, 20 mM MES (pH 5.6), 10 mM CaCl₂, and 0.1% BSA. Leaves were roughly chopped with a clean razor in a petri dish, then allowed to incubate in the digestion solution for 4 h on a shaker. After digestion, the solution was filtered using 100 μm cell filters, transferred to a mL centrifuge tube and centrifuged for 2 min at 100 RCF. The supernatant fluid was carefully pipetted and discarded. The protoplasts were resuspended in 2 mL of W5 buffer solution, then allowed to precipitate on ice for 30 min, after which the supernatant was removed and replaced with a MMg solution (0.4 M mannitol, 15 mM MgCl₂, 4 mM MES, pH 5.6) to stabilize the cells.

### Confocal Microscopy of Control and CNC-Treated Arabidopsis Leaves, Tobacco Cells and Protoplasts

Leaves of 12-day-old A. thaliana seedlings were soaked in water (control), DTAF solution, CNC solution, or DTAF-CNC solution (1 mg/mL) for 4 hours and then intensively washed with ddH₂O to fully remove residual treatments. Both freshly isolated N. tabacum protoplasts and callus cells that were cultivated for 20 days on standard Murashige and Skoog medium (MS medium) were immersed into 1 mg/mL of FITC-CNC conjugate or solution CNC and allowed to incubate for 4 hours. Samples were then thoroughly washed by consecutively centrifuging the samples, removing the supernatant, and resuspending in MMG solution comprised 0.5 M D-mannitol, 15 mM MgCl₂, and 4 mM MES (pH=5.7) three times. Image acquisition of treated tobacco cells was done using an AxioObserver LSM 880 (Zeiss International, Germany) equipped with a 63 x/1.4 oil objective located within the digital microscopy laboratory at UAMS (University of Arkansas for Medical Sciences, Little Rock, AR). The laser wavelength and power chosen for FITC excitation was 488 nm and 11.8% respectively. DTAF imaging was done at 488 nm with 15.8 power. Image processing and analysis of fluorescent intensity was accomplished using Zeiss ZEN 3.5 software (Zeiss International, Germany). Statistical analysis of fluorescent intensity was completed using SPSS software with a one-way ANOVA, α=0.05.

### Experiments with Tobacco Cell Culture and CNC-2,4-D Conjugates

N. tabacum callus culture that was established by Khodakovskaya et al. was used in experiments. Callus aggregates (300 mg inoculum) were seeded in individual glass tubes (Phytotechnology Laboratories, Inc.) containing 25 mL of MS medium with and without supplements and cultivated in controlled environmental conditions (dark, 22-24 C) for 35 days. 10 replicates were used for each treatment. Callus growth was on the following mediums was assessed: MS medium (no 2,4-D supplement); MS medium supplemented with reduced dose of 2,4-D (0.4 mg/L); MS medium supplemented only with CNC (0.4 mg/L); MS medium supplemented with non-conjugated CNC (0.4 mg/L) and 2,4-D (0.4 mg/L); and MS medium supplemented with CNC-2,4-D conjugate (with 0.4 mg/L 2,4D). Total produced cell biomass was evaluated by measurement of total fresh weight of callus culture generated in the end of cultivation period.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications, and various publications, are cited and discussed in the description of this invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

### REFERENCE LIST

1. IPPC Secretariat. Scientific review of the impact of climate change on plant pests. (FAO on behalf of the IPPC Secretariat, 2021). doi:10.4060/cb4769en.
2. Di Baldassarre, G. et al. Water shortages worsened by reservoir effects. Nat Sustain 1, 617-622 (2018).
3. United Nations Department of Economic and Social Affairs, Population Division. World Population Prospects 2022: Summary of Results. (2022).
4. Shang, Y. et al. Applications of Nanotechnology in Plant Growth and Crop Protection: A Review. Molecules 24, 2558 (2019).
5. Giraldo, J. P., Wu, H., Newkirk, G. M. & Kruss, S. Nanobiotechnology approaches for engineering smart plant sensors. Nat. Nanotechnol. 14, 541-553 (2019).
6. Pandey, K. et al. Effects of carbon-based nanomaterials on seed germination, biomass accumulation and salt stress response of bioenergy crops. PLOS ONE 13, e0202274 (2018).
7. Lowry, G. V., Avellan, A. & Gilbertson, L. M. Opportunities and challenges for nanotechnology in the agri-tech revolution. Nat. Nanotechnol. 14, 517-522 (2019).
8. Mujtaba, M. et al. Nanocarrier-Mediated Delivery of miRNA, RNAi, and CRISPR-Cas for Plant Protection: Current Trends and Future Directions. ACS Agric. Sci. Technol. 1, 417-435 (2021).
9. Khodakovskaya, M. V. et al. Carbon Nanotubes as Plant Growth Regulators: Effects on Tomato Growth, Reproductive System, and Soil Microbial Community. Small 9, 115-123 (2013).
10. Demirer, G. S. et al. High aspect ratio nanomaterials enable delivery of functional genetic material without DNA integration in mature plants. Nat. Nanotechnol. 14, 456-464 (2019).
11. Sylvester, D. J., Abbott, K. W. & Marchant, G. E. Not again! Public perception, regulation, and nanotechnology. Regulation & Governance 3, 165-185 (2009).
12. Khodakovskaya, M. V. et al. Complex genetic, photothermal, and photoacoustic analysis of nanoparticle-plant interactions. Proceedings of the National Academy of Sciences 108, 1028-1033 (2011).
13. Rezaei Cherati, S. et al. Comprehensive Risk Assessment of Carbon Nanotubes Used for Agricultural Applications. ACS Nano 16,12061-12072 (2022).
14. Perez-de-Luque, A. Interaction of Nanomaterials with Plants: What Do We Need for Real Applications in Agriculture? Frontiers in Environmental Science 5, (2017).
15. Guilherme, M. R. et al. Pectin-based polymer hydrogel as a carrier for release of agricultural nutrients and removal of heavy metals from wastewater. J. Appl. Polym. Sci. n/a-n/a (2010) doi:10.1002/app.32123.
16. Hanna, E. A. et al. Zein Nanoparticles for Enhanced Translocation of Pesticide in Soybean (Glycine max). ACS Agric. Sci. Technol. (2022) doi:10.1021/acsagscitech.2c00160.
17. Machado, T. O. et al. Bio-Based Lignin Nanocarriers Loaded with Fungicides as a Versatile Platform for Drug Delivery in Plants. Biomacromolecules 21, 2755-2763 (2020).
18. Huang, S., Zhou, L., Li, M.-C., Wu, Q. & Zhou, D. Cellulose Nanocrystals (CNCs) from Corn Stalk: Activation Energy Analysis. Materials 10, (2017).
19. Demirer, G. S. et al. High aspect ratio nanomaterials enable delivery of functional genetic material without DNA integration in mature plants. Nat. Nanotechnol. 14, 456-464 (2019).
20. Serag, M. F. et al. Trafficking and Subcellular Localization of Multiwalled Carbon Nanotubes in Plant Cells. ACS Nano 5, 493-499 (2011).
21. Blanco, A. et al. Chapter 5 - Nanocellulose for Industrial Use: Cellulose Nanofibers (CNF), Cellulose Nanocrystals (CNC), and Bacterial Cellulose (BC). in Handbook of Nanomaterials for Industrial Applications (ed. Mustansar Hussain, C.) 74-126 (Elsevier, 2018). doi:10.1016/B978-0-12-813351-4.00005-5.
22. Beck-Candanedo, S., Roman, M. & Gray, D. G. Effect of Reaction Conditions on the Properties and Behavior of Wood Cellulose Nanocrystal Suspensions. Biomacromolecules 6, 1048-1054 (2005).
23. George, J. & Sabapathi, S. Cellulose nanocrystals: synthesis, functional properties, and applications. Nanotechnol Sci Appl 8, 45-54 (2015).
24. Xiao, Y. et al. Cellulose nanocrystals prepared from wheat bran: Characterization and cytotoxicity assessment. International Journal of Biological Macromolecules 140, 225-233 (2019).
25. Hazman Danial, W., Taib, R. M., Samah, M. A. A., Salim, R. M. & Majid, Z. A. The valorization of municipal grass waste for the extraction of cellulose nanocrystals. RSC Advances 10, 42400-42407 (2020).
26. Abdelaziz, R. M., El-Maghraby, A., Sadik, W. A.-A., El-Demerdash, A.-G. M. & Fadl, E. A. Biodegradable cellulose nanocrystals hydrogels for removal of acid red 8 dye from aqueous solutions. Sci Rep 12, 6424 (2022).
27. Lee, S., Jeong, J. H., Kim, S. H. & Shin, H. Freezing Tolerance Enhancement and Thermographic Observation of Whole Peach Trees Applied with Cellulose Nanocrystals under Realistic Spring Frost Conditions Using a Soil-Fruit-Daylit-System. Plants 10, 2301 (2021).
28. Arnoldussen, B. et al. Internal freezing and heat loss of apple (Malus domestica Borkh.)

and sweet cherry (Prunus avium L.) reproductive buds are decreased with cellulose nanocrystal dispersions. Frontiers in Plant Science 13, (2022).
29. Elabasy, A., Shoaib, A., Waqas, M., Shi, Z. & Jiang, M. Cellulose Nanocrystals Loaded with Thiamethoxam: Fabrication, Characterization, and Evaluation of Insecticidal Activity against Phenacoccus solenopsis Tinsley (Hemiptera: Pseudococcidae). Nanomaterials 10, 788 (2020).
30. Roman, M. Toxicity of Cellulose Nanocrystals: A Review. Industrial Biotechnology 11, 25-33 (2015).
31. Hosseinidoust, Z., Alam, M. N., Sim, G., Tufenkji, N. & van de Ven, T. G. M. Cellulose nanocrystals with tunable surface charge for nanomedicine. Nanoscale 7, 16647-16657 (2015).
32. Tizzotti, M., Charlot, A., Fleury, E., Stenzel, M. & Bernard, J. Modification of Polysaccharides Through Controlled/Living Radical Polymerization Grafting—Towards the Generation of High Performance Hybrids. Macromolecular Rapid Communications 31, 1751-1772 (2010).
33. Meng, X. & Edgar, K. J. “Click” reactions in polysaccharide modification. Progress in Polymer Science 53, 52-85 (2016).
34. Pimentel, D. Amounts of pesticides reaching target pests: Environmental impacts and ethics. J Agric Environ Ethics 8, 17-29 (1995).
35. Duke, S. O. Pesticide Dose—A Parameter with Many Implications. in Pesticide Dose: Effects on the Environment and Target and Non-Target Organisms vol. 1249 1-13 (American Chemical Society, 2017).
36. Nutrition, C. for F. S. and A. GRAS Substances (SCOGS) Database—Select Committee on GRAS Substances (SCOGS) Opinion: Cellulose, Microcrystalline cellulose. http://wayback.archive-it.org/7993/20171031063405/https://www.fda.gov/Food/IngredientsPackagingLabeling/GRAS/SCOGS/ucm261287.htm.
37. Khodakovskaya, M. V., de Silva, K., Bilis, A. S., Dervishi, E. & Villagarcia, H. Carbon Nanotubes Induce Growth Enhancement of Tobacco Cells. ACS Nano 6, 2128-2135 (2012).
38. Lin, N. & Dufresne, A. Nanocellulose in biomedicine: Current status and future prospect. European Polymer Journal 59, 302-325 (2014).
39. Jorfi, M. & Foster, E. J. Recent advances in nanocellulose for biomedical applications. Journal of Applied Polymer Science 132, (2015).
40. Liebert, T., Kostag, M., Wotschadlo, J. & Heinze, T. Stable Cellulose Nanospheres for Cellular Uptake. Macromolecular Bioscience 11, 1387-1392 (2011).
41. Mahmoud, K. A. et al. Effect of Surface Charge on the Cellular Uptake and Cytotoxicity of Fluorescent Labeled Cellulose Nanocrystals. ACS Appl. Mater. Interfaces 2, 2924-2932 (2010).
42. Ortiz-Matamoros, M. F., Villanueva, M. A. & Islas-Flores, T. Genetic transformation of cell-walled plant and algae cells: delivering DNA through the cell wall. Briefings in Functional Genomics 17, 26-33 (2018).
43. Abitbol, T., Palermo, A., Moran-Mirabal, J. M. & Cranston, E. D. Fluorescent Labeling and Characterization of Cellulose Nanocrystals with Varying Charge Contents. Biomacromolecules 14, 3278-3284 (2013).
44. Dong, S., Cho, H. J., Lee, Y. W. & Roman, M. Synthesis and Cellular Uptake of Folic Acid-Conjugated Cellulose Nanocrystals for Cancer Targeting. Biomacromolecules 15, 1560-1567 (2014).
45. Eyley, S. & Thielemans, W. Surface modification of cellulose nanocrystals. Nanoscale 6, 7764-7779 (2014).
46. Singh, R. K. & Khatri, 0. P. A scanning electron microscope based new method for determining degree of substitution of sodium carboxymethyl cellulose. J Microsc 246, 43-52 (2012).
47. Davey, M. R., Anthony, P., Patel, D. & Power, J. B. Plant Protoplasts: Isolation, Culture and Plant Regeneration. in Plant Cell Culture 153-173 (John Wiley & Sons, Ltd, 2010). doi:10.1002/9780470686522.ch9.
48. Freisthler, M. S. et al. Association between increasing agricultural use of 2,4-D and population biomarkers of exposure: findings from the National Health and Nutrition Examination Survey, 2001-2014. Environmental Health 21, 23 (2022).
49. Wang, Y. et al. Quantitative Analysis of Compatibility and Dispersibility in Nanocellulose-Reinforced Composites: Hansen Solubility and Raman Mapping. ACS Nano 15, 20148-20163 (2021).
50. Sternberg, L. O., Deniro, M. J. & Johnson, H. B. Isotope Ratios of Cellulose from Plants Having Different Photosynthetic Pathways. Plant Physiology 74, 557-561 (1984).
51. Nair, R. et al. Nanoparticulate material delivery to plants. Plant Science 179, 154-163 (2010).
52. Ghormade, V., Deshpande, M. V. & Paknikar, K. M. Perspectives for nano-biotechnology enabled protection and nutrition of plants. Biotechnology Advances 29, 792-803 (2011).
53. Mwaanga, P. Risks, Uncertainties, and Ethics of Nanotechnology in Agriculture. New Visions in Plant Science (IntechOpen, 2018). doi:10.5772/intechopen.76590.
54. Délye, C., Jasieniuk, M. & Le Corre, V. Deciphering the evolution of herbicide resistance in weeds. Trends in Genetics 29, 649-658 (2013).
55. Sosnoskie, L. M., Culpepper, A. S., Braxton, L. B. & Richburg, J. S. Evaluating the Volatility of Three Formulations of 2,4-D When Applied in the Field. Weed Technology 29, 177-184 (2015).
56. Lombardo, S. et al. Thermodynamic Study of the Interaction of Bovine Serum Albumin and Amino Acids with Cellulose Nanocrystals. Langmuir 33, 5473-5481 (2017).
57. Lahiani, M. H., Dervishi, E., Ivanov, I., Chen, J. & Khodakovskaya, M. Comparative study of plant responses to carbon-based nanomaterials with different morphologies. Nanotechnology 27, 265102 (2016).
58. Lahiani, M. H. et al. Impact of Carbon Nanotube Exposure to Seeds of Valuable Crops. ACS Appl. Mater. Interfaces 5, 7965-7973 (2013).
59. Schneider, C. A., Rasband, W. S. & Eliceiri, K. W. NIH Image to ImageJ. 25 years of image analysis. Nat Methods 9, 671-675 (2012).
60. Du, X., Smirnov, A., Pluskal, T., Jia, W. & Sumner, S. Metabolomics Data Preprocessing Using ADAP and MZmine 2. in Computational Methods and Data Analysis for Metabolomics (ed. Li, S.) 25-48 (Springer US, 2020). doi:10.1007/978-1-0716-0239-3_3.
61. Ogata, H. et al. KEGG: Kyoto Encyclopedia of Genes and Genomes. Nucleic Acids Res 27, 29-34 (1999).
62. Xia, J., Sinelnikov, I. V., Han, B. & Wishart, D. S. MetaboAnalyst 3.0—making metabolomics more meaningful. Nucleic Acids Res 43, W251-W257 (2015).

What is claimed is:

1. A nanomaterial conjugate for delivering an active agent to a plant, the nanomaterial conjugate comprising:
a nanocarrier; and
an active agent linked to the nanocarrier,
wherein the nanocarrier is cellulose nanocrystal (CNC); and
wherein the nanocarrier delivers the active agent across cell walls of cells of the plant.

2. The nanomaterial conjugate of claim 1, wherein
the active agent comprises an agrochemical.

3. The nanomaterial conjugate of claim 1, wherein
the active agent comprises a growth regulator.

4. The nanomaterial conjugate of claim 2, wherein
the agrochemical is 2,4-dichlorophenoxyacetic acid (2,4-D).

5. The nanomaterial conjugate of claim 1, wherein
the active agent comprises a large molecule.

6. The nanomaterial conjugate of claim 5, wherein
the large molecule comprises a polynucleotide.

7. The nanomaterial conjugate of claim 5, wherein the large molecule comprises a polypeptide.

8. The nanomaterial conjugate of claim 1, wherein
effective dose of the nanomaterial conjugate is lower than effective dose of the active agent alone.

9. The nanomaterial conjugate of claim 1, wherein
the nanocarrier and the active agent are covalently linked.

10. The nanomaterial conjugate of claim 1, wherein
the nanocarrier and the active agent are non-covalently linked.

11. The nanomaterial conjugate of claim 1, wherein
the nanocarrier is not toxic to the plant at a concentration below 100 g/L.

12. The nanomaterial conjugate of claim 1, wherein
the nanomaterial conjugate is not toxic to the plant at a concentration below 500 mg/L.

13. A method for delivering an active agent across cell walls of plant cells, comprising
providing the plant cells in a medium;
introducing an amount of a nanomaterial conjugate to the medium to form a mixture thereof; and
maintaining the mixture at a temperature for a period of time to allow sufficient interaction of the plant cells with the nanomaterial conjugate,
wherein the nanomaterial conjugate comprises a nanocarrier and an active agent linked to the nanocarrier;
wherein the nanocarrier is cellulose natural crystal; and
wherein the nanocarrier delivers the active agent across the cell walls of the plant cells.

14. The nanomaterial conjugate of claim 13, wherein
the active agent comprises an agrochemical.

15. The nanomaterial conjugate of claim 14, wherein
the growth regulator is 2,4-dichlorophenoxyacetic acid (2,4-D).

16. The nanomaterial conjugate of claim 13, wherein
the active agent comprises a large molecule.

17. The nanomaterial conjugate of claim 13, wherein
effective dose of the nanomaterial conjugate is lower than effective dose of the active agent alone.

18. The nanomaterial conjugate of claim 17, wherein
the effective dose of the nanomaterial conjugate is less than 50% of the effective dose of the active agent alone.

19. The nanomaterial conjugate of claim 13, wherein
the nanomaterial conjugate is applied at a concentration in a range of 0.1 mg/L to 500 mg/L.
